(19) United States
(12) Patent Application Publication
(10) Pub. No.: US 2020/0237557 A1
(43) Pub. Date: Jul. 30, 2020

(54) CATARACTOUS LENS PULVERIZER

(71) Applicant: Shu-Fong Ou, Taipei (TW)

(72) Inventor: Shu-Fong Ou, Taipei (TW)

(73) Assignee: Universsal Vision Biotechnology Co., Ltd.

(21) Appl. No.: 16/258,697

(22) Filed: Jan. 28, 2019

(51) Int. Cl. A61F 9/007

(52) U.S. Cl. CPC A61F 9/00745; A61F 9/00754

(57) ABSTRACT

A cataract lens nucleus breaker for eye surgery, comprising a tube body, an operating rod and a draw ring; the tube body is a hollow tube having a tapered portion and a thin tube portion extending outwardly from a front end thereof, and an opening portion at a rear end thereof; an edge of the opening portion has an operating portion, the tapered portion has a venting hole; the operating rod has a front end disposed in the tube body and a rear end protruding from the opening portion; the draw ring is formed by a wire having a connecting portion connected to the front end of the operating rod, and a nucleus breaking ring; the nucleus breaking ring is housed in the tapered portion and can be pushed and pulled back to the taper portion by the operating rod.

### BACKGROUND OF INVENTION

### 1. Field of the Invention

The present invention relates generally to a cataractous lens pulverizer for cataract surgery.

### 2. Description of Related Art

When. the soluble protein changes into insoluble protein gradually for any reason, the crystalline lens becomes opaque, leading to blurred vision, this is known as cataract; the aging is the most familiar cause, the other factors include diabetes, injuries, inflammation and heredity.

Once the crystalline lens of the eye becomes opaque, it gradually causes hypopsia, and turns opaque white eventually, thus preventing the light from entering the eye, and, the patient loses vision. Up to now, there is not yet a pharmacotherapy proved effective on preventing or curing cataract.

At present, the cataract surgery is the unique effective curing method. There are two kinds of operation, one is “ultrasonic phacoemulsification and intraocular lens implantation”, which is a small incision cataract surgery, the crystalline lens is emulsified by ultrasonic and sucked away, the wound is free of suturing or sutured, by only one to two stitches; the other one is “extracapsular cataract extraction and intraocular lens implantation”, adopted when the cataract is hypermature, a large wound is required for extracting the whole lens nucleus, so the wound shall be sutured by 6 to 7 stitches; a special lens or a contact lens is required for correction after operation, so as to recover the patient's vision,

In a surgical operation using ultrasonic phacoemulsification to remove the crystalline lens, the crystalline grain shall be crushed before emulsification, so the crystalline grain is crushed by using the cataract grain crushing device for eye surgery shown in the applicant's previous patent document 1. There is no problem in using the device of patent document 1, but the cost of the medical appliance is high, mostly reused, and a high learning cost is derived from the formative education for medical students, unsuitable for discarding, leading to sanitary safety problems.

In view of this, how to solve the problems is the topic to be improved by the present invention.

Patent document 1: applicant's previous project, U.S. Pat. No. 6,554,843, “Cataract instrument”.

### SUMMARY OF THE INVENTION

The purpose of the present invention is to provide a cataractous lens pulverizer easy to operate for eye surgery.

In order to solve the above technical problems, the present invention adopts the following technical solutions:

A cataractous lens pulverizer for eye surgery, comprising:

a tube body, an operating rod and a draw ring;

wherein the tube body is a hollow tube having a tapered portion and a thin tube portion extending outwardly from a front end thereof, and an opening portion at a rear end thereof; an edge of the opening portion has an operating portion, the tapered portion has a venting hole;

wherein the operating rod has a front end disposed in the tube body and a rear end protruding from the opening portion;

wherein the draw ring is formed by a wire, which has a connecting portion connected to the front end of the operating rod, and a nucleus breaking ring disposed at a free end of the connecting portion; the nucleus breaking ring is housed in the tapered portion and can be pushed and pulled back to the taper portion by the operating rod.

More particularly, wherein the operating rod has a rod body disposed in the tube body and movable forward and backward toward the opening portion, a front end of the rod, body has a rod head disposed in the tube body, and an annular portion is provided at a rear end thereof.

More particularly, wherein the operating rod further includes a collar is disposed at a joint between the rod head and the rod body; the collar is made of a soft material and has an outer circumference corresponding to the thin tube portion, and has an axle hole for the rod head to pass through.

More particularly, wherein one end of the nucleus breaking ring is connected to the free end, of the connecting portion, and another other end thereof is not connected to the front end of the operating rod, or connected to the front end of the operating rod.

More particularly, wherein the wire has a circular in cross section and is made of non-metallic or metal.

More particularly, wherein the rod body is further includes a blocking piece at one end of the collar and a plurality of reinforcing ribs looped therearound and disposed between the blocking piece and the annular portion.

The following results can be obtained according to the implementation method:

The cataractous lens pulverizer of the present invention maintains the original function of cataractous lens pulverizer by the coordination of tube, operating rod and draw ring, the cataract grain can be cut by using the draw ring for subsequent ultrasonic phacoemulsification to remove the grain. The present invention can reduce the medical treatment cost, so that the doctors spend less mind on maintenance, and the structural design approximating a syringe is easy for a novice to learn and operate, and the learning cost is reduce, it can be abandoned after use, it is sanitary and safe.

### BRIEF DESCRIPTION OF THE DRAWINGS

FIG. 1 is a perspective view of a cataractous lens pulverizer of the present invention;

FIG.2. is an exploded perspective view of the cataractous lens pulverizer of the present invention;

FIG.3 is a schematic cross-sectional view of the cataractous lens pulverizer of the present invention;

FIG A is a schematic view of the cataractous lens pulverizer applied to cataract crystal pulverization;

FIG. 5 is an exploded perspective view of the cataractous lens pulverizer in combination with another type of draw ring.

### DETAILED DESCRIPTION OF THE INVENTION

The detailed description is given below according to the figures:

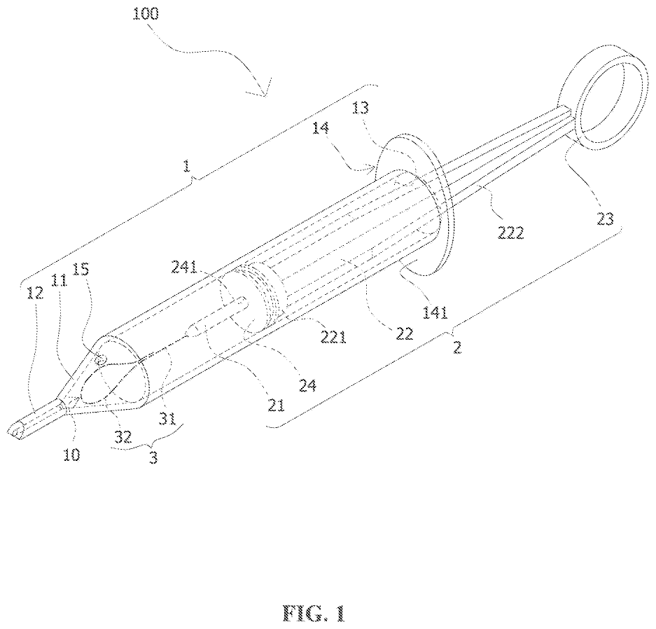

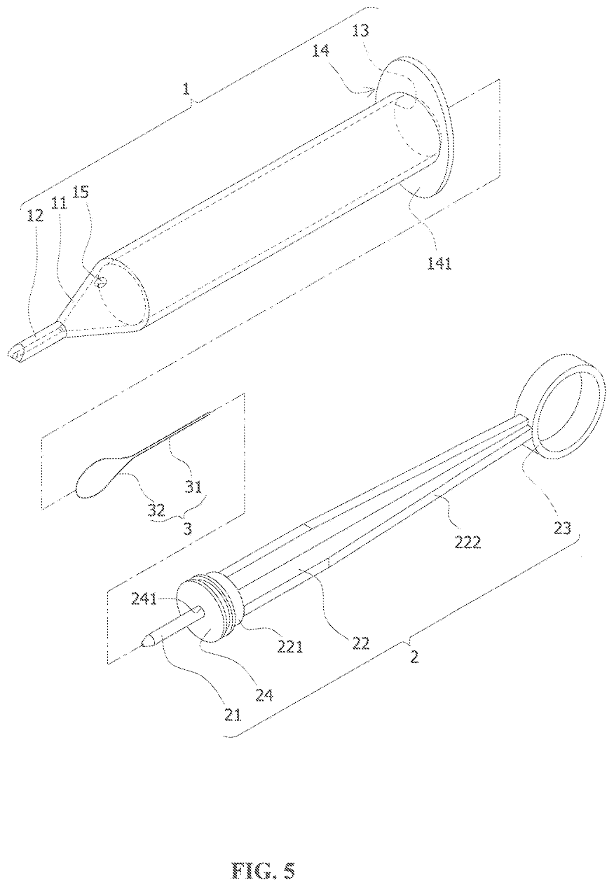

FIGS. 1˜3 and FIG.5 disclose a cataractous lens pulverizer, the cataractous lens pulverizer 100 is used for eye surgery, comprising a tube body 1, an, operating rod 2 and a draw ring 3. The feature is that the tube body 1 is hollow, and its front end is provided with a tapered portion 11 and a thin tube portion 12 from inside to outside, its back end is provided with an opening portion 13, an operating portion 14 is located at the edge of the opening portion 13, the tapered portion 11 is provided with an venting hole 15; the front end of the operating rod 2 is located in the tube body 1, the back end extends out of the opening portion 13; the draw ring 3 is made by rolling a filament 10, forming a connecting portion 31 connected to the front end of the operating rod 2, and a collar-shaped nucleus breaking ring 32 located at the free end of the connecting portion 31, and the nucleus breaking ring 32 is stored in the tapered portion 11, and it can be pushed out of or drawn back into the tapered portion 11 by the operating rod 2.

The original function of the cataractous lens pulverizer of the previous project is maintained by the coordination of the tube body 1, operating rod 2 and draw ring 3, and the cataractous lens pulverizer 100 of the present invention reduces the cost of medical materials, so that the doctors' learning cost is reduced, and the maintenance cost and mental efforts for maintenance are reduced.

Secondly, the design of tapered portion 11 and thin tube portion 12 is favorable for cataract grain crushing operation, please see FIG. 4, the combination of operating rod 2 and tube body 1 makes the overall cataractous lens pulverizer 100 as easy as a syringe to be operated, the draw ring 3 is used together with operating rod 2 to crush the grain.

Furthermore, the tube body 1 can be operated and held flexibly by the operator, the operating rod 2 pushes the draw ring 3 out of thin tube portion 12 from the tapered portion 11, so as to ring the crystalline lens or the cataract grain; and the operating rod 2 draws back the draw ring 3 into the tapered portion 11 through the thin tube portion 12, a crimping force is generated to crush the cataract grain. The cataractous lens pulverizer 100 of the present invention can implement the cataract grain crushing device with the least components.

In addition, the venting hole 15 avoids injecting air into the eye, the air in the front end of tube body 1 can be discharged smoothly when using the operating rod 2, the use is safe, the operating rod 2 is pushed smoothly.

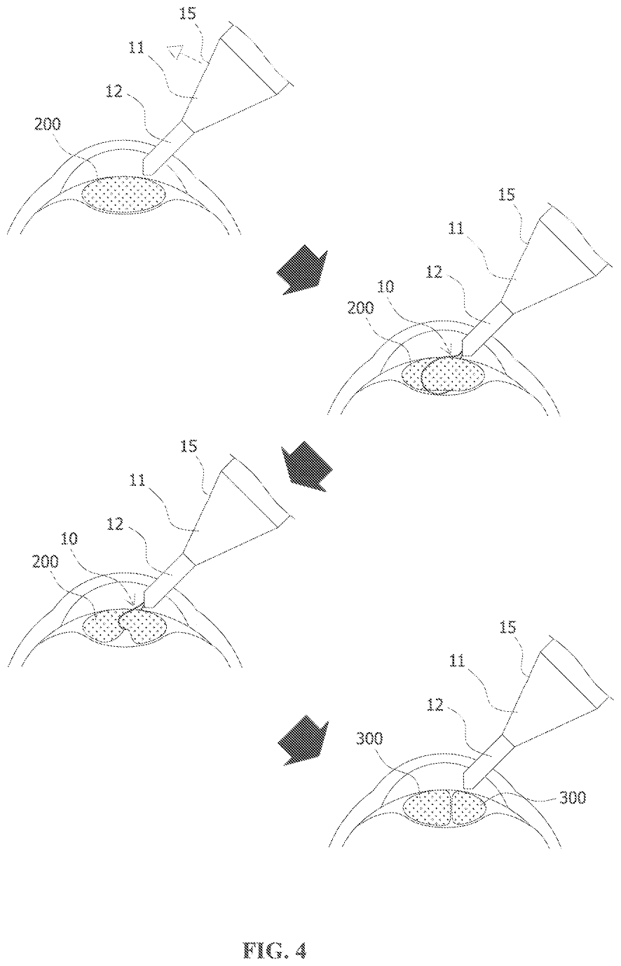

As shown in FIGS. 3˜4, before the user applies a force to the operating rod 2, the draw ring 3 at the front end of operating rod 2 is located in the tapered portion 11, the operating rod 2 moves forward when it is pushed, the draw ring 3 is deformed and extended out of the thin tube portion 12 through the tapered portion 11 and thin tube portion 12, so as to ring the crystalline lens 200, Afterwards, the operating rod 2 is pulled to draw back the draw ring 3, a crimping force if generated while the filament 10 is drawn back into the tapered portion 11 from the thin tube portion 12, so the crystalline lens 200 can be cut up into two halves smoothly, forming two grains 300.

Afterwards, the action is repeated [not disclosed in the figure], the two grains 300 can be split up one by one, this process is performed continuously, the grain 300 is cut up into more smaller grains, till the sizes of small grains 300 are smaller than the emulsified size defined by ultrasonic phacoemulsification, the grains 300 are removed by ultrasonic phacoemulsification.

As shown in FIG2, the operating portion 14 is a flange plate 141; the front end of the operating rod 2 has a rod head 21 located in tube body 1, and the back end has a rod body 22 connected to the rod head 21 and the free end thereof can extend out of the opening portion 13, the free end of the rod body 22 is provided with a annular portion 23 for the finger to go through.

The flange plate 141 facilitates applying force and holding; the annular portion 23 is convenient for the finger to apply force, easy of handling, and the coordination of flange plate 141 and annular portion 23 makes the cataractous lens pulverizer 100 of the present invention convenient to be used and implemented,

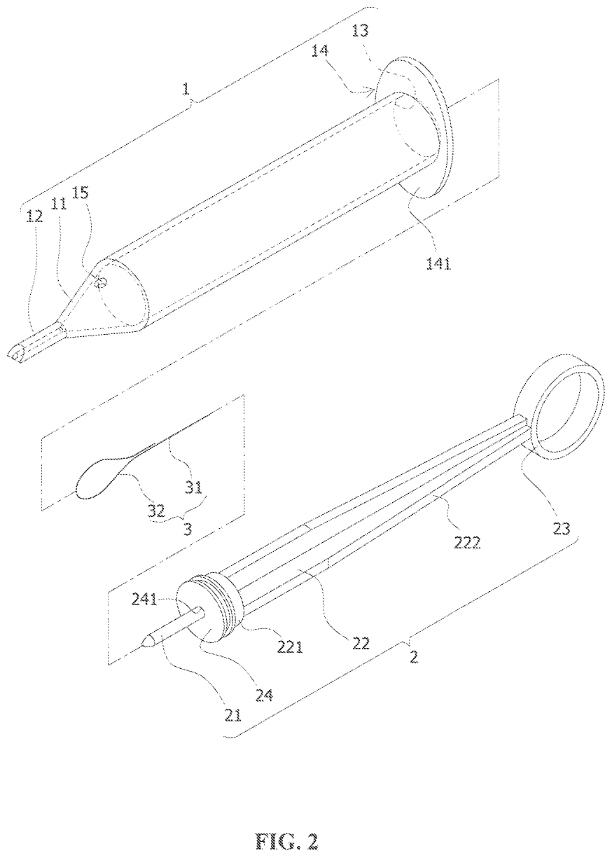

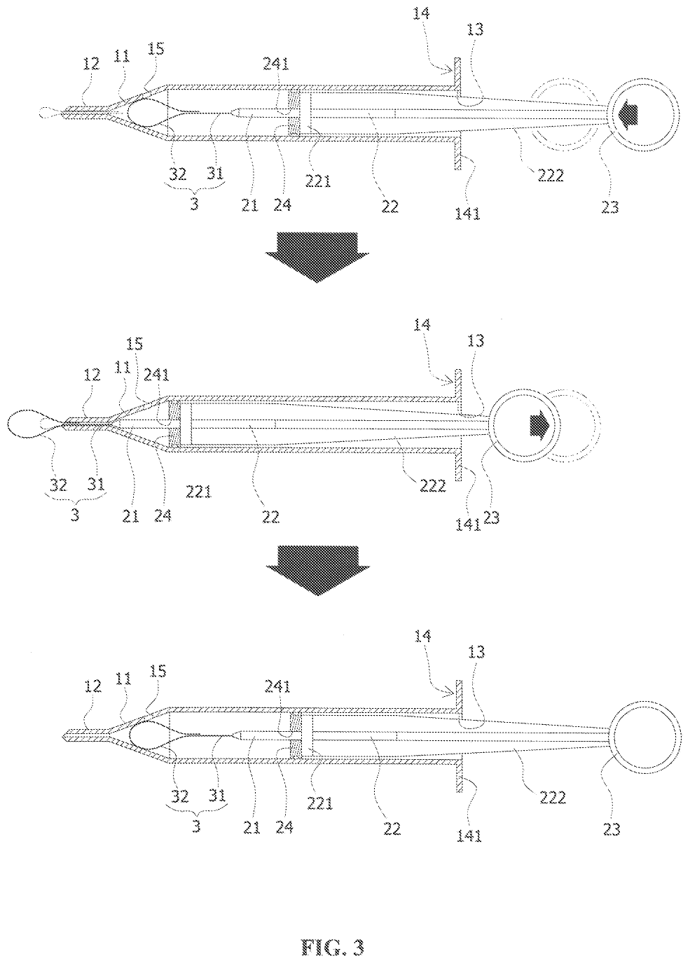

As shown in FIG.2, the operating rod 2 has a collar 24 located at the joint of the rod head 21 and the rod body 22. The collar 24 is made of a soft material, the outside diameter corresponds to tube body 1, and it has an axle hole 241 for the rod head 21 to go through.

The collar 24 enables the operating rod 2 to move in the tube body 1 smoothly, and avoids shaking influencing the safety of operation.

As shown in FIG. 2 and FIG. 5, one end of the nucleus breaking ring 32 is connected to the free end of the connecting portion 31, and the other end is unconnected to the front end of the operating rod 2 or connected to the front end of the operating rod 2.

Different nucleus breaking rings 32 are used according to the user's requirement, convenient for ringing and pulling the whole opaque crystalline lens, so as to cut the crystalline lens.

The section of t e filament 10 is circular, and the filament can be nonmetal or metal.

The design of the circular section can avoid excessive pressure of nucleus breaking ring 32 damaging the patient's eyeball when the operator is performing an operation on the patient, and the draw ring 3 can bend in any direction, favorable for the user to operate.

Secondly, different filaments 10 are used according to the user's requirement for medical behavior. If the filament 10 is a nonmetal filament, it can be abandoned after use, meeting the medical requirement for one-time use, If the filament 10 is a metal filament, it is sterilized after use to meet the medical requirement for reuse.

In addition, the metal filament can be memory filament or stainless steel alloy filament, the memory filament is a better choice, it is unlikely to be deformed permanently by extrusion when passing through the tapered portion 11 of tube body 1.

As shown FIG. 2 and FIG. 5, one end of the rod body 22 corresponding to the collar 24 is provided with a blocking piece 221, multiple reinforcing ribs 222 are located annularly between the blocking piece 221 and the annular portion 23, The blocking piece 221 provides a supporting force which is favorable for smooth movement when the collar 24 is pushed, and the reinforcing ribs 222 make the overall structure steady and safe.

The above only describes some exemplary embodiments of the present invention. Those having ordinary skills in the art may also make many modifications and improvements without departing from the conception of the invention, which shall all fall within the protection scope of the invention.

1. A cataractous lens pulverizer for eye surgery, comprising:
a tube body (1), an operating rod (2) and a draw ring (3);
wherein the tube body (1) is a hollow tube having a tapered portion (11) and a thin tube portion (12) extending outwardly from a front end thereof, and an opening portion (13) at a rear end thereof; an edge of the opening portion (13) has an operating portion (14), the tapered portion (11) has a venting hole (15);
wherein the operating rod (2) has a front end disposed in the tube body (1) and a rear end protruding from the opening portion (13);
wherein the draw ring (3) is formed by a wire (10), which has a connecting portion (31) connected to the front end of the operating rod (2), and a nucleus breaking ring (32) disposed at a free end of the connecting portion (31); the nucleus breaking ring (32) is housed in the tapered portion (11) and can be pushed and pulled back to the taper portion (11) by the operating rod (2).

2. The cataractous lens pulverizer according to claim 1, wherein the operating rod (2) has a rod, body (22) disposed in the tube body (1) and movable forward and backward toward the opening portion (13), a front end of the rod body (22) has a rod head (21) disposed in the tube body (1), and an annular portion (23) is provided at a rear end thereof.

3. The cataractous lens pulverizer according to claim 2, wherein the operating rod (2) further includes a collar (24) disposed at a joint between the rod head (21) and the rod body (22), the collar (24) is made of a soft material and has an outer circumference corresponding to the thin tube portion (12), and has an axle hole (241) for the rod head (21) to pass through,

4. The cataractous lens pulverizer according to claim 1, wherein one end of the nucleus breaking ring (32) is connected to the free end of the connecting portion (31), and another other end thereof is not connected to the front end of the operating rod (2), or connected to the front end of the operating rod (2).

5. The cataractous lens pulverizer according to claim 1, wherein the wire (10) has a circular in cross section and is made of non-metallic or metal.

6. The cataractous lens pulverizer according to claim 2, wherein the rod body (22) is further includes a blocking piece (221) at one end of the collar (24) and a plurality of reinforcing ribs (222) looped therearound and disposed bet ween the blocking piece (221) and the annular portion (23).
